(19) United States
(12) Patent Application Publication
(10) Pub. No.: US 2020/0200726 A1
(43) Pub. Date: Jun. 25, 2020

(54) WATER QUALITY MEASUREMENT DEVICE

(71) Applicant: KURITA WATER INDUSTRIES LTD., Tokyo (JP)

(72) Inventor: Yoshimasa NAKANO, Tokyo (JP)

(21) Appl. No.: 16/494,092

(22) PCT Filed: Sep. 12, 2017
(86) PCT No.: PCT/JP2017/032799

§ 371 Date: Sep. 13, 2019

(30) Foreign Application Priority Data
Mar. 27, 2017 (JP) 2017-061396

(51) Int. Cl. G01N 33/18; C02F 1/00

(52) U.S. Cl. CPC G01N 33/18; C02F 1/00

(57) ABSTRACT

When the water quality of effluent water or intermediate water is measured, the effluent water or the intermediate water is allowed to flow from a pipe 40, 50, or 60 to a measurement column 84 via pipes 37, 80, 83, and the water quality is measured by a sensor 85. In a case where the water quality of raw water is measured, the effluent water and the raw water are introduced from pipes 40, 70 and mixed in a line mixer 38, and the water quality is measured by the sensor 85. After the measurement of the water quality, the effluent water (or fresh water) is allowed to flow, to clean the sensor 85 and the like, and the water quality of the effluent water (or fresh water) is measured to check the characteristics of the sensor 85.

### TECHNICAL FIELD

The present invention relates to a water quality measurement device, and particularly relates to a water quality measurement device suitable for measuring the water quality of water with a high concentration of a substance to be measured, such as water discharged from a process for biologically treating organic wastewater.

### BACKGROUND ART

In a case where a quality of water to be treated in wastewater treatment equipment is measured by a sensor, there has been used a conductivity meter, an absorptiometer, a pH meter, an oxidation-reduction potential (ORP) meter, or the like (Patent Literature 1).

In a case where the water qualities of more than one types of sample water are measured, the following has been performed: an on-off valve is provided in each of a plurality of pipes provided so as to allow each type of sample water to flow into a measurement unit, each on-off valve is opened sequentially, and the water quality of each type of sample water is measured sequentially (Patent Literature 2).

PTL1: JP 2014-004550 A

PTL2: JPH 08-082581 A

In a continuous measurement device for measuring the water qualities of a plurality of types of test liquid while continuously switching the test liquid, when the concentrations of the respective types of test liquid vary greatly, for example, when liquid with a conductivity of about 1 mS/m is measured after liquid with a conductivity of about 100 mS/m, the response of a conductivity meter is delayed, and hence it is necessary to leave a certain amount of time until the output of a sensor becomes stable. In a case where the water quality is continuously measured while the test liquid is continuously switched, when the time until the output stability operation is obtained increases, a sampling period increases by the number of types of switched test liquid.

For reducing a measurement error due to a variation over time in detection characteristics of a measurement unit, it is necessary to perform calibration at some intervals, but the calibration practice requires a temporary interruption of the measurement, which becomes an obstacle when a plurality of types of test liquid are to be measured continuously.

### SUMMARY OF INVENTION

An object of the present invention is to provide a water quality measurement device capable of efficiently and accurately performing water quality measurement also in the case of a plurality of types of sample water that vary greatly in concentration of a substance to be measured.

A water quality measurement device of the present invention includes: a plurality of sample water lines; a clean water line; a confluence line in which each of the sample water lines and the clean water line are continuous to each other via a switching means; a valve for sample water, provided in each of the sample water lines; a valve for clean water, provided in the clean water line; a water quality measurement unit that measures properties of water from the confluence line; and a control means that controls the switching means so as to communicate the clean water line to the confluence line before and/or after (i.e. at least one of before and after) any one of the sample water lines is connected to the confluence line and water quality measurement for sample water is performed.

One aspect of the present invention further includes a monitoring device that monitors a water quality measurement value of clean water in a case where the clean water line is communicated to the confluence line and the clean water is supplied to the water quality measurement unit.

### Advantageous Effects of Invention

In the water quality measurement device of the present invention, in a case where the sample water to be supplied to the water quality measurement unit is switched, prior to the supply of the sample water after the switching, clean water is supplied to the water quality measurement unit to temporarily immerse the water quality measurement unit in the clean water (liquid with low concentrations of various components), thereby initializing the water quality measurement unit to a constant state.

In the present invention, after the supply of the clean water to the water quality measurement unit, the operation of the water quality measurement unit to return to the initial state is monitored, to evaluate the responsiveness of the water quality measurement unit. When the return of the water quality measurement unit to the initial state is not completed within a pre-set time, the transmission, display, or the like of a signal is performed, the signal indicating the need for the calibration, repair, replacement, or the like of the water quality measurement unit.

### BRIEF DESCRIPTION OF DRAWINGS

FIG. 1 is a configuration diagram of a water quality measurement device according to an embodiment.

FIG. 2 is a configuration diagram of a water treatment apparatus.

### DESCRIPTION OF EMBODIMENT

Hereinafter, an embodiment will be described with reference to the drawings.

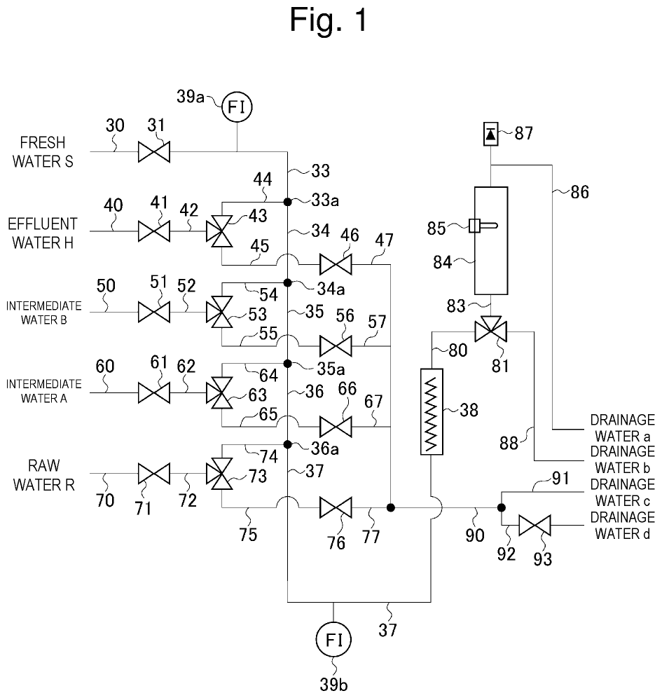

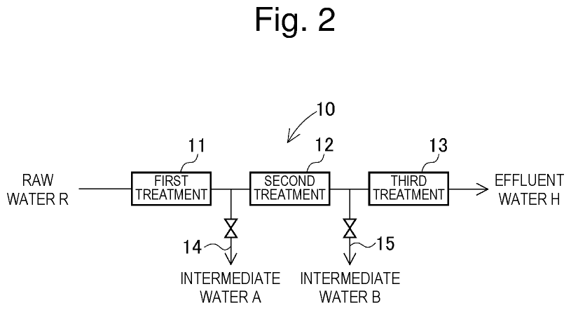

FIG. 1 is a configuration diagram of a water quality measurement device according to the embodiment. The water quality measurement device is for measuring the water qualities of raw water, intermediate water A, B, and effluent water in a drainage treatment apparatus 10 shown in FIG. 2.

The drainage treatment apparatus 10 is configured to treat raw water in a first treatment device (e.g., aerobic biological treatment device) 11, a second treatment device (e.g., pressure floatation device) 12, and a third treatment device (e.g., filter) 13, and release the treated water. First treated water flowing out of the first treatment device 11 is collected via a collection line 14 as intermediate water A, and the second treated water flowing out from the second treatment device 12 is collected via the collection line 15 as intermediate water B.

The water quality measurement device of FIG. 1 is able to measure the water quality of the effluent water and is also able to measure the water quality of each of the intermediate water A, B and the raw water by dilution with clean fresh water S. The water quality measurement device can clean a flow path and a sensor with the fresh water S or the effluent water H. As the fresh water S, it is possible to use tap water, well water, or other industrial water.

The fresh water S can be supplied to a line mixer 38 via a pipe 30 (dilution water line), a valve 31, a pipe 33, a T-shaped joint 33a, pipe 34, a T-shaped joint 34a, a pipe 35, a T-shaped joint 35a, a pipe 36, a T-shaped joint 36a, and a pipe 37. The pipes 33, 37 are provided with flow meters 39a, 39b. The pipes 34 to 37 constitute a confluence line.

A pipe (sample water line) 40 for supplying the effluent water H is connected to the T-shaped joint 33a via a valve 41, a pipe 42, a three-way valve 43, and a pipe 44 continuous to one outlet of the three-way valve 43. The other outlet of the three-way valve 43 is continuous to a pipe 90 via a pipe 45, a valve 46, and a pipe 47.

A pipe (sample water line) 50 for supplying the intermediate water B is connected to the T-shaped joint 34a via a valve 51, a pipe 52, a three-way valve 53, and a pipe 54 continuous to one outlet of the three-way valve 53. The other outlet of the three-way valve 53 is continuous to the pipe (drainage water line) 90 via a pipe 55, a valve 56, and a pipe 57.

A pipe (sample water line) 60 for supplying the intermediate water A is connected to the T-shaped joint 35a via a valve 61, a pipe 62, a three-way valve 63, and a pipe 64 continuous to one outlet of the three-way valve 63. The other outlet of the three-way valve 63 is continuous to the pipe 90 via a pipe 65, a valve 66, and a pipe 67.

A pipe (sample water line) 70 for supplying raw water R is connected to the T-shaped joint 36a via a valve 71, a pipe 72, a three-way valve 73, and a pipe 74 continuous to one outlet of the three-way valve 73. The other outlet of the three-way valve 73 is continuous to the pipe 90 via a pipe 75, a valve 76, and a pipe 77.

The line mixer 38 on the outflow side is continuous to the inlet of the measurement column 84 via a pipe 80, a three-way valve 81, and a pipe 83 continuous to one outlet of the three-way valve 81. The measurement column 84 is provided with one water quality sensor 85, or two or more water quality sensors 85, such as an electrical conductivity meter, an absorptiometer, a pH meter, or an ORP meter. Here, as the absorptiometer, it is possible to use an organic monitor (ultraviolet (UV) meter) that measures the degree of organic contamination in the sample water (organic matter) as the absorbance (light absorption degree) of ultraviolet rays. Then, a pipe 86 for allowing drainage water a to flow out is connected to the outlet of the measurement column 84.

The pipe 86 near the measurement column 84 is connected with an intake valve 87 that allows air to flow into the measurement column 84 at the time of discharging the water in the measurement column 84 from a pipe 88 to be described later.

The pipe 88 for discharging drainage water b is connected to the other outlet of the three-way valve 81.

The pipe 90 branches into the pipes 91, 92 and discharges drainage water c, d through the respective pipes. The pipe 92 is provided with a valve 93.

The valve 71 is for flow rate regulation and has a predetermined opening. Each of the other valves or each of the three-way valves is opened/closed or caused to switch the flow path by a control device (not shown) in accordance with a predetermined sequence. Then, except for the case of inspection, maintenance, or the like, normally, the valves 41, 51, 61 are open and the valves 46, 56, 66, 76, 93 are closed, and the valves are in these states in the following as well.

A description will be given of a method for measuring the water qualities of the effluent water H, the intermediate water A, B, and the raw water R with the water quality measurement device.

In the initial state, the valve 31 and the intake valve 87 are closed. The three-way valves 43, 53, 63, 73, 81 communicate between the pipes 42 and 45, between the pipes 52 and 55, between the pipes 62 and 65, between the pipes 72 and 75, and between the pipes 80 and 88. When the start switch of the control device is pressed, the control device first allows only the effluent water H to flow through the measurement column 84, to perform the water quality measurement of the effluent water.

In the water quality measurement for the effluent water, the three-way valve 43 is brought into the state of communicating between the pipes 42 and 44, the valve 31 is closed, and the three-way valves 53, 63, 73 are caused to communicate between the pipes 52 and 55, between the pipes 62 and 65, and between the pipes 72 and 75. Further, the three-way valve 81 is brought into the state of communicating between the pipes 80 and 83. Thereby, the effluent water H flows sequentially through the pipes 40, 42, 44, 34 to 37, the line mixer 38, and the pipes 80, 83. When all the water in the measurement column 84 becomes the effluent water H, and a predetermined time further elapses, the water quality of the effluent water H is measured by the sensor 85 of the measurement column 84.

Due to a sufficiently low concentration of a dissolved substance in the effluent water H, by first introducing the effluent water H to the measurement column 84, the sensor 85 is initialized to a constant state.

When the water quality is measured by the sensor 85, the three-way valve 81 may be switched so as to communicate between the pipes 80 and 88, to bring the water in the measurement column 84 into a stopped state, or the measurement may be performed with the sensor 85 while the effluent water H is kept flowing through the measurement column 84. This also applies to the time of the water quality measurement for the intermediate water B or A or the raw water R to be described later.

After the water quality measurement for the effluent water H, the valve 31 is opened in a state where the three-way valve 43 communicates between the pipes 42 and 45 and the inflow of the effluent water H is stopped. The fresh water S flows sequentially through the pipes 30, 33 to 37, the line mixer 38, the pipes 80, 83, the measurement column 84, and the pipe 86, to initialize the sensor 85 while cleaning the inside system for a predetermined time.

At this time, the temporal change in the output value of the sensor 85 is monitored by the monitoring device to evaluate the responsiveness of the sensor 85. When the return of the sensor 85 to the initial state is not completed within a pre-set time (e.g., when the output value does not decrease to a value obtained at the time of the past water quality measurement for fresh water), the transmission, display, or the like of a signal is performed, the signal indicating the need for the calibration, repair, replacement, or the like of the sensor 85.

When the sensor 85 is evaluated as normal, such as a case where the output value of the sensor 85 returns to the past water quality value of the fresh water within a predetermined time, the valve 31 is then closed to stop the flow of the fresh water S into the pipes 33 to 37.

In that state, the intake valve 87 is opened and the three-way valve 81 is caused to communicate between the pipes 83 and 88, to discharge the water in the measurement column 84 as the drainage water b. After the discharge of the drainage water b has been completed, the intake valve 87 is closed, and the three-way valve 81 is brought into the state of communicating between the pipes 80 and 83.

Next, in order to perform the water quality measurement for the intermediate water B, the three-way valve 53 is caused to communicate between the pipes 52 and 54. The intermediate water B flows sequentially through the pipes 50, 52, 54, 35 to 37, the line mixer 38, the pipes 80, 83, the measurement column 84, and the pipe 86. When the intermediate water B is allowed to flow for a predetermined time, so that all the water in the measurement column 84 becomes the intermediate water B, and a predetermined time further elapses, the water quality of the intermediate water B is measured by the sensor 85 of the measurement column 84.

After the water quality measurement for the intermediate water B, the valve 31 is opened in a state where the three-way valve 53 communicates between the pipes 52 and 55 and the inflow of the intermediate water B is stopped. The fresh water S flows sequentially through the pipes 30, 33 to 37, the line mixer 38, the pipes 80, 83, the measurement column 84, and the pipe 86, to initialize the sensor 85 while cleaning the inside system for a predetermined time.

At this time, the temporal change in the output value of the sensor 85 is monitored by the monitoring device to evaluate the responsiveness of the sensor 85. When the return of the sensor 85 to the initial state is not completed within a pre-set time (e.g., when the output value does not decrease to a value obtained at the time of the past water quality measurement for fresh water), the transmission, display, or the like of a signal is performed, the signal indicating the need for the calibration, repair, replacement, or the like of the sensor 85.

When the sensor 85 is evaluated as normal, such as a case where the output value of the sensor 85 returns to the past water quality value of the fresh water within a predetermined time, the valve 31 is then closed to stop the flow of the fresh water S into the pipes 33 to 37.

In that state, the three-way valve 81 is caused to communicate between the pipes 83 and 88 while the intake valve 87 is opened, to discharge the water in the measurement column 84 as the drainage water b. After the discharge of the drainage water b has been completed, the intake valve 87 is closed, and the three-way valve 81 is brought into the state of communicating between the pipes 80 and 83.

Subsequently, in order to perform the water quality measurement for the intermediate water A, the three-way valve 63 is caused to communicate between the pipes 62 and 64. The intermediate water A flows sequentially through the pipes 60, 62, 64, 36, 37, the line mixer 38, the pipes 80, 83, the measurement column 84, and the pipe 86. When the intermediate water A is allowed to flow for a predetermined time, so that all the water in the measurement column 84 becomes the intermediate water A, and a predetermined time further elapses, the water quality of the intermediate water A is measured by the sensor 85 of the measurement column 84.

After the water quality measurement for the intermediate water A, the valve 31 is opened in a state where the three-way valve 63 communicates between the pipes 62 and 65 and the inflow of the intermediate water A is stopped. The fresh water S flows sequentially through the pipes 30, 33 to 37, the line mixer 38, the pipes 80, 83, the measurement column 84, and the pipe 86, to initialize the sensor 85 while cleaning the inside system for a predetermined time.

At this time, the time course of the output value of the sensor 85 is monitored by the monitoring device to evaluate the responsiveness of the sensor 85. When the return of the sensor 85 to the initial state is not completed within a pre-set time, the transmission, display, or the like of a signal is performed, the signal indicating the need for the calibration, repair, replacement, or the like of the sensor 85.

When the sensor 85 is evaluated as normal, thereafter, the valve 31 is closed to stop the flow of the fresh water S into the pipes 33 to 37. In that state, the three-way valve 81 is caused to communicate between the pipes 83 and 88 while the intake valve 87 is opened, to discharge the water in the measurement column 84 as the drainage water b. After the discharge of the drainage water b has been completed, the intake valve 87 is closed, and the three-way valve 81 is brought into the state of communicating between the pipes 80 and 83.

Subsequently, the measurement shifts to the water quality measurement for the raw water R. In a case where the water quality of the raw water R is measured, in order to prevent fresh water with a high concentration from directly coming into contact with the sensor 85 to contaminate the sensor and from affecting the sensitivity characteristics, the raw water R is diluted with the fresh water S and allowed to flow through a sensor column. That is, the valve 31 is opened, and the fresh water S is allowed to flow through the pipes 30, 33 to 37, the line mixer 38, the pipes 80, 83, the measurement column 84, and the pipe 86. Further, the three-way valve 73 is caused to communicate between the pipes 72 and 74 while the valve 31 is opened, and the raw water R is added to the pipe 37 via the T-shaped joint 36a. Thereby, the raw water R and the fresh water S pass through the pipe 37 and are mixed sufficiently in the line mixer 38 to become diluted raw water. When the diluted raw water is allowed to flow for a predetermined time, so that all the water in the measurement column 84 becomes the diluted raw water, and a predetermined time further elapses, the water quality of the diluted raw water is measured by the sensor 85.

The flow rate of the fresh water S at the time of performing the above dilution is detected by the flow meter 39a. The total flow rate of the fresh water S and the raw water R is detected by the flow meter 39b. The flow rate of the raw water R is the difference in detected flow rate between the flow meters 39b and 39a, so that the flow rate and the dilution ratio of the raw water are determined from each detected flow rate. The water quality of the raw water is calculated based on the detected values of the sensor 85 for the fresh water S and the diluted raw water (the past value is used as the detected value for fresh water) and the dilution ratio. Note that it is possible to monitor the clogging of each valve and each pipe based on the detected value of the flow meter 39b. Further, the dilution ratio can be regulated by regulating the opening of the valve 31.

After the completion of the water quality measurement for the raw water R, the three-way valve 73 is caused to communicate between the pipes 72 and 75, to stop the flow of the raw water R into the pipe 37, and only the fresh water S is allowed to flow through the pipes 30, 33 to 37, the line mixer 38, the pipes 80, 83, the measurement column 84, and the pipe 86, to initialize the sensor 85 while cleaning these flow paths and the sensor 85.

At this time, the time course of the output value of the sensor 85 is monitored by the monitoring device to evaluate the responsiveness of the sensor 85. When the return of the sensor 85 to the initial state is not completed within a pre-set time, the transmission, display, or the like of a signal is performed, the signal indicating the need for the calibration, repair, replacement, or the like of the sensor 85. When the sensor 85 is evaluated as normal, thereafter, the valve 31 is closed to stop the flow of the fresh water S into the pipes 33 to 37.

In that state, the three-way valve 81 is caused to communicate between the pipes 83 and 88 while the intake valve 87 is opened, to discharge the water in the measurement column 84 as the drainage water b. After the discharge of the drainage water b has been completed, the intake valve 87 is closed, and the three-way valve 81 is brought into the state of communicating between the pipes 80 and 83.

Next, the valve 31 is opened so that the water quality measurement for the fresh water S is performed. The fresh water S flows sequentially through the pipes 30, 33 to 37, the line mixer 38, the pipes 80, 83, the measurement column 84, and the pipe 86. When the fresh water S is allowed to flow for a predetermined time, so that all the water in the measurement column 84 becomes the fresh water S, and a predetermined time further elapses, the water quality of the fresh water S is measured by the sensor 85 of the measurement column 84. Then, the waiting time is taken until the next water quality measurement.

During the waiting time until the next water quality measurement, the fresh water S may be held (retained) in the flow path or may be allowed to flow continuously through the pipes 30, 33 to 37, the line mixer 38, the pipes 80, 83, the measurement column 84, and the pipe 86.

In the above description, only the raw water R has been diluted with the fresh water S, the intermediate water A, B may also be diluted if necessary.

In the above description, the fresh water S has been used as the clean water, and after the water quality measurement for the effluent water H, the intermediate water B or A, or the raw water R, the fresh water S has been allowed to flow, to clean the flow path and the sensor 85 and perform the initialization and the responsiveness evaluation of the sensor 85. However, the effluent water H may be used instead of the fresh water S, and after the water quality measurement for the intermediate water B or A, or the raw water R, the effluent water H may be allowed to flow, to clean the flow path and the sensor 85 and perform the initialization and the responsiveness evaluation of the sensor 85.

In the above description, in the case where the intermediate water B or A or the raw water R is allowed to flow through the pipe 35, 36 or 37 from each pipe 50, 60 or 70a, the three-way valve 53, 63, or 73 has been simply switched so as to communicate between the pipes 52 and 54, between the pipes 62 and 64, or the pipe 72, 74. However, after being discharged from the pipes 90, 91 via the pipes 55, 57, the pipes 65, 67, or the pipes 75, 77 for a predetermined time, the intermediate water B or A or the raw water R is allowed to flow through the pipes 35, 36, or 37 by switching the three-way valve 53, 63, or 73 as described above.

For performing this operation, the valves 56, 66, or 76 is opened while the three-way valve 53, 63 or 73 is caused to communicate between the pipes 52 and 55, between the pipes 62 and 65, or between the pipes 72 and 75, to allow the intermediate water B or A or the raw water R to flow out of the pipe 91. At this time, the valve 51, 61, or 71 may be fully opened to allow the intermediate water B or A or the raw water R to flow for a predetermined time, and thereafter, the opening of the valve 51, 61, or 71 may be narrowed. Then, the three-way valve 53, 63 or 73 is caused to communicate between the pipes 52 and 54, between the pipes 62 and 64, or between the pipes 72 and 74, and the valve 56, 66 or 76 is closed. By fully opening the valve 51, 61, or 71 as thus described, the intermediate water B or A or the raw water R remaining in the pipe 50, 60, or 70 can be discharged from the pipe 90, 91, and the fresh intermediate water B or A or the raw water R just collected from the water treatment apparatus 10 can be taken into the water quality measurement device in an early stage. Further, an increase in the flow rate by fully opening the valve 51, 61, or 71 can prevent the clogging of the pipes and valves (especially, the valve 71).

In the present invention, the fresh intermediate water B may be taken into the pipe 50 as described above while the water quality measurement for the effluent water H is being performed, the fresh intermediate water A may be taken into the pipe 60 as described above while the water quality measurement for the intermediate water B is being performed, and the fresh raw water R may be taken into the pipe 70 as described above while the water quality measurement for the intermediate water A is being performed.

When the concentration of suspended matter in the raw water R is high, it is desirable to use a full-bore valve as the valve 71 and prevent the clogging of the valve 71. The full-bore valve may also be used for the other valves.

In the above description, the water quality measurement has been performed on the four types of water, the raw water, the intermediate water A, B, and the effluent water H by using the four pipes 40, 50, 60, 70, but by decreasing or increasing the number of pipes, one, two, or three types of water, or five types or more of water, can be diluted and subjected to the water quality measurement.

In the above description, it has been assumed that the dilution is performed with the fresh water S, but the dilution may be performed with the effluent water H. In this case, the flow meter 39a is provided in the pipe 34 so as to determine a ratio of dilution with the effluent water H. When dilution water or cleaning water is used as the effluent water H, the water supply cost or industrial water cost can be reduced.

In the water quality measurement device of FIG. 1, since the water qualities of the effluent water H, intermediate water A, B, and the raw water R are measured using the same sensor 85, even if there is a variation in the measurement sensitivity, the zero point, or the like of the sensor 85, it is possible to relatively evaluate the water qualities of the effluent water H, the intermediate water A, B, and the raw water R by making a comparison between the water quality measurement values of the effluent water H, intermediate water A, B, and the raw water R or between these values and the water quality measurement value of the fresh water S.

The water quality measurement device of FIG. 1 is suitable for being used in water quality measurement for raw water and final treated water, as well as water under treatment discharged from each treatment process, in treatment facilities for organic drainage water such as food plant drainage water. The water quality measurement device of FIG. 1 is particularly suitable for being used in the water quality measurement for drainage water with a high organic concentration, in which biochemical oxygen demand (BOD) of raw water is 200 mg/L or more, for example, about 1000 to 5000 mg/L, and for intermediate treatment water of the drainage water.

While the present invention has been described in detail with a specific embodiment, it is clear to those skilled in the art that various changes can be made without deviating from the intention and the scope of the invention.

This application is based on Japanese Patent Application 2017-061396 filed on Mar. 27, 2017, and the whole is incorporated by reference.

### REFERENCE SIGNS LIST

11 first treatment device

12 second treatment device

13 third treatment device

38 line mixer

39a, 39b flow meter

84 measurement column

85 water quality sensor

1. A water quality measurement device comprising:
a plurality of sample water lines;
a clean water line;
a confluence line in which each of the sample water lines and the clean water line are continuous to each other via a switching means;
a valve for sample water, provided in each of the sample water lines;
a valve for clean water, provided in the clean water line;
a water quality measurement unit that measures properties of water from the confluence line; and
a control means that controls the switching means so as to communicate the clean water line to the confluence line before and/or after any one of the sample water lines is connected to the confluence line and water quality measurement for sample water is performed.

2. The water quality measurement device according to claim 1, further comprising
a monitoring device that monitors a water quality measurement value of clean water in a case where the clean water line is communicated to the confluence line and the clean water is supplied to the water quality measurement unit.
